(19) United States
(12) Patent Application Publication
(10) Pub. No.: US 2009/0018454 A1
(43) Pub. Date: Jan. 15, 2009

(54) Finger-type pulse detection wireless transmission structure

(76) Inventor: Chin-Yeh Hung, Taoyuan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/822,883

(22) Filed: Jul. 11, 2007

(51) Int. Cl. A61B 5/02

(52) U.S. Cl. 600/500

(57) ABSTRACT

A finger-type pulse detection wireless transmission structure, comprising: a) a housing made according to a finger's contour, a first lug and a second lug being formed at both sides of the housing, an infrared pulse sensor being disposed at the bottom of the housing for detecting the change of a slight amount of blood within the blood vessel of a user's finger that is caused by the pulsation such that the heartbeat pulse signal of the user is available; b) a sensitive switch disposed at the bottom of the housing for turning power on and off the infrared pulse sensor; c) a finger fixing band having a first end connected to the first lug and a second end passing through the second lug, the finger fixing band being stuck in an reverse direction to the external side such that the housing can be bound to the finger while the infrared pulse sensor and the sensitive switch are in close contact to the finger's skin; d) a noise-canceling circuit and a filtering shaping amplifying circuit installed within the housing and electrically coupled to the infrared pulse sensor; and e) a radio frequency module having a wireless signal transmission circuit and provided within the housing for transmitting the heartbeat pulse signal of the filtering shaping amplifying circuit to a display storage unit provided with a wireless signal receiving circuit such that the heartbeat pulse signal can be displayed.

### BACKGROUND OF THE INVENTION

1. Field of the Invention

The invention relates to a pulse detector, and more particularly, to a structure having a detection unit put on a finger while the heartbeat signal is wirelessly transmitted to a display storage unit for achieving an easy use and an accurate measurement of the heartbeat.

2. Description of the Related Art

Conventionally, people's heart rate remarkably varies before and after people take exercise. During exercise, measuring the variables is very important. To take exercise properly and advantageously, the heart rate must increase to 60% of the maximum rate; however, if the heart rate increases to the maximum rate during exercise, it will do harm to the exerciser. Thus, a user-friendly and precise heart rate measurement device is very important for taking exercise to keep fit.

Next, FIG. 1 shows an electrode belt of heart rate monitor disclosed in U.S. Pat. No. 6,553,247, in which a heart rate monitoring unit 60 is bound on the chest with a belt 61 to monitor the heart rate on and on and wirelessly transmit a signal to a receiver 70 bound around the wrist. However, it is found that the patent is poor with the following conditions when being applied.

1. In general, the heart rate monitoring unit 60 is tightly bound with the belt 61 on the chest to keep close to; however, the lung capacity increases during exercise and thus the belt bound up on the chest causes a user to be uncomfortable and breathe hard.

2. Female chest contour is bigger, so the heart rate monitoring unit 60 cannot stay fully close to the chest, thereby the accuracy of monitoring decreasing.

3. To accurately measure the heart rate, the heart rate monitoring unit 70 cannot be bound outside the clothes but inside the clothes and close to the chest, so the belt 71 is placed certainly after the clothes is taken off, which might be somewhat inconvenient to males but may be implemented none the less; however, it is very inconvenient to females to bind up the belt 71 in a public place; thus, the conventional belt is not suitable for the female users.

In addition, there is a conventional heartbeat sensor disposed on a wristwatch. The user may place his fingertip or his thumb on the heartbeat sensor for measuring his heartbeat. However, this conventional unit is not suitable for use during the exercise sessions since the large movement will change the detected signals. Meanwhile, the finger has to stay on the sensor in a compressed way for a problemfree measurement. This is very impractical.

Consequently, how to minimize the size of the heartbeat detector for an easy use and an accurate measurement is the main topic of the invention.

### SUMMARY OF THE INVENTION

It is a main object of the invention to provide a finger-type pulse detection wireless transmission structure having a pulse detector that is minimized in size for binding onto a finger. Thus, the structure in accordance with the invention is light, convenient, and burdenfree. Meanwhile, it is also suitable for the female users to use without affecting their movement.

It is another object of the invention to provide a finger-type pulse detection wireless transmission structure having a pulse detector in close contact to the finger's skin without worrying about any undesired slip or displacement. Thus, an accurate measurement of the tiny change of the reflected infrared caused by the blood flowing action within the blood vessel each time due to the pulsation is ensured. Accordingly, the accuracy in measurement can be improved.

It is a further object of the invention to provide a finger-type pulse detection wireless transmission structure that permits an automatic power on when putting on the finger and an automatic power off when removed. In this way, the consumption of the electric power can be reduced for prolonging its service life.

### BRIEF DESCRIPTION OF THE FIGURES

The accomplishment of this and other objects of the invention will become apparent from the following descriptions and its accompanying figures of which:

FIG. 1 is a schematic view illustrating the structure of a device disclosed in U.S. Pat. No. 6,553,247;

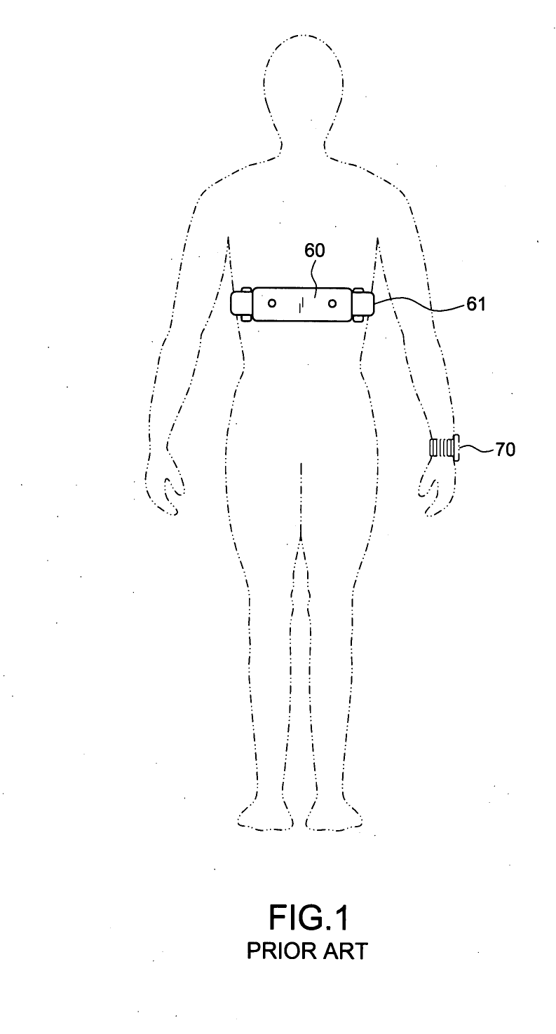

FIGS. 2A and 2B are perspective views of the front and rear side of a pulse detector in accordance with the invention, respectively;

FIG. 3 is a perspective view of the pulse detector of the invention bound to a finger;

FIG. 4 is a circuit block diagram of a preferred embodiment according to the invention; and

FIG. 5 is a perspective view of an application example of the invention; and

FIG. 6 is a perspective view of another application example of the invention.

### DETAILED DESCRIPTION OF THE PREFERRED EMBODIMENT

Now, the present invention will be described more specifically with reference to the following embodiments. It is to be noted that the following descriptions of preferred embodiments of the invention are presented herein for purpose of illustration and description only; it is not intended to be exhaustive or to be limited to the precise form disclosed.

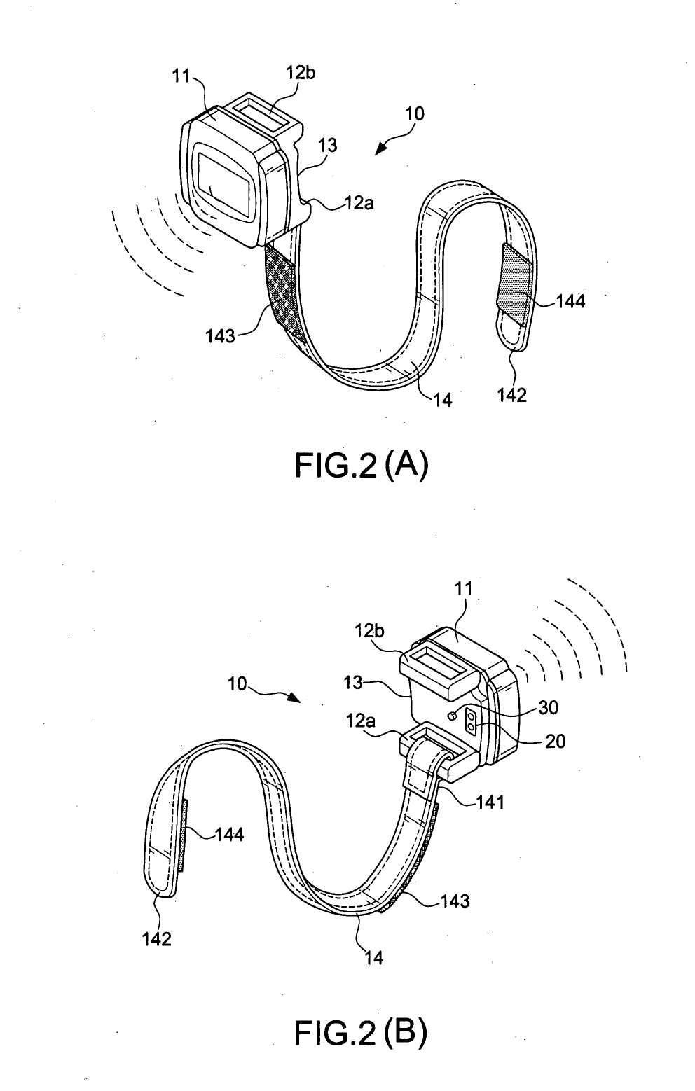

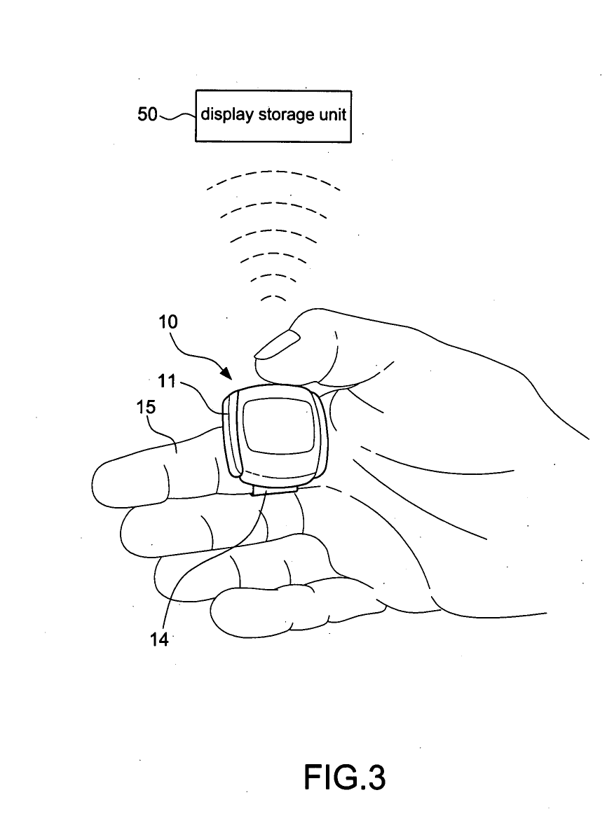

First, with reference to FIGS. 2A, 2B, 3, and 4, the invention mainly includes a finger-type pulse detector 10 and a display storage unit 50 separate therefrom. The finger-type pulse detector 10 includes a housing 11, a sensitive switch 30, a finger fixing band 14, a noise-canceling circuit 32, a filtering shaping amplifying circuit 33, and a radio frequency module 40.

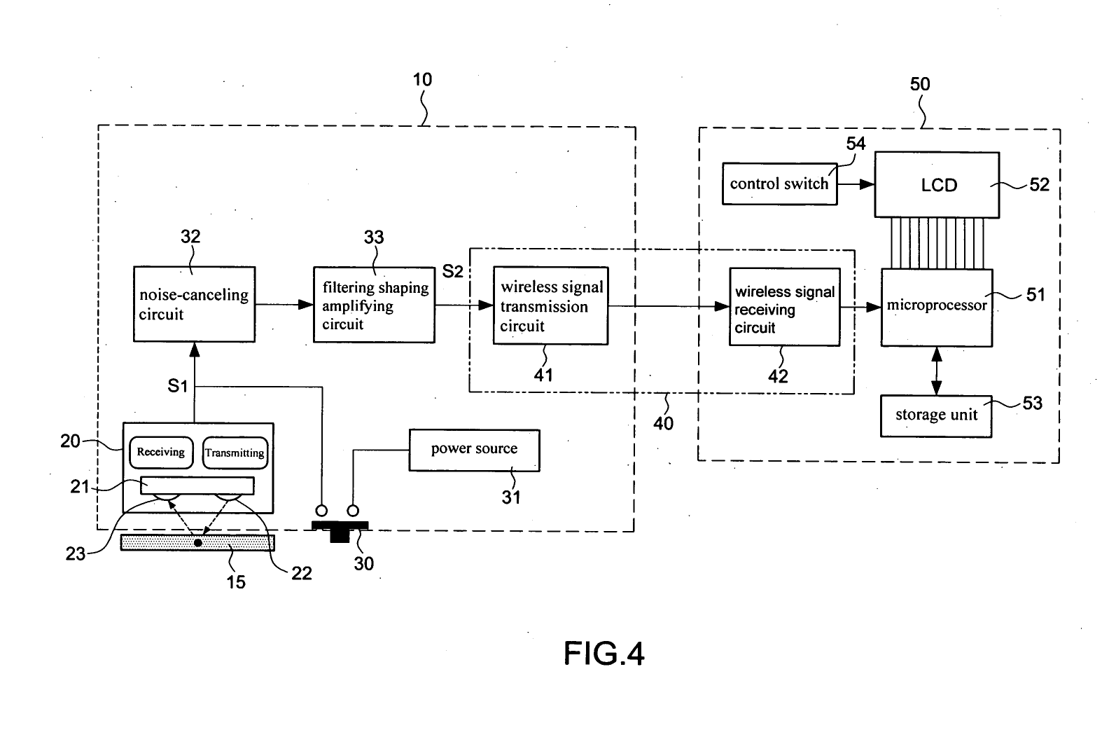

The housing 11 is made according to the finger's contour. A first lug 12a and a second lug 12b are formed at both sides of the housing 11. An infrared pulse sensor 20 is disposed at the bottom of the housing 11 for detecting the change of a slight amount of blood within the blood vessel of a user's finger 15 that is caused by the pulsation. As shown in FIG. 4, the infrared pulse sensor 20 includes an infrared transmitting unit 22 disposed on a control circuit board 21 and a receiving unit 23 arranged in parallel thereto. According to this embodiment, the infrared transmitting unit 22 is an infrared light-emitting diode while the receiving unit 23 is a receiving transistor. The user's heartbeat pulse signal is available by means that the reflection signal of the emitted infrared passing through the blood vessel is received by the transistor.

According to the embodiment, the bottom of the housing 11 is substantially formed as a concave side 13 corresponding to the curvature of a finger. In this way, the concave side 13 can be in close contact to the finger. This ensures a comfortable wearing. Moreover, the closer the concave side 13 approaches to the skin and blood vessel, the more accurate the detection is.

The sensitive switch 30 is disposed at the bottom of the housing 11 for turning power on and off the infrared pulse sensor 20. According to this embodiment, the sensitive switch 30 is positioned at the flank of the infrared pulse sensor 20 (see FIG. 4), but should not be restricted thereto. In other words, the sensitive switch 30 may be disposed at the center of the infrared pulse sensor 20, that is, at the center between the infrared transmitting unit 22 and the receiving unit 23. Anyway, the most important is that an automatic heartbeat detection takes place when the bottom of the housing 11 is in contact to the finger 15. Meanwhile, an automatic disconnection is done when the housing 11 is removed form the finger 15. Thus, the consumption of the power source 31 can be reduced for prolonging the service life of the products.

The finger fixing band 14 has a first end 141 connected to the first lug 12a and a second end 142 passing through the second lug 12b and stuck in a reverse direction to the external side. In this way, the housing 11 can be bound to the finger 15 in such a way that the infrared pulse sensor 20 and the sensitive switch 30 are in close contact to the finger's skin. The finger fixing band 14 includes a Velcro fastening consisting of tiny nylon hooks 143 near an external side of the first end 141 and a dense nylon pile 144 at an internal side of the second end 142 such that the tiny nylon hooks 143 and the dense nylon pile 144 interlock when pressed together.

As shown in FIG. 4, a noise-canceling circuit 32 and a filtering shaping amplifying circuit 33 are installed within the housing 11 and electrically coupled to the infrared pulse sensor 20. The human body pulse signal obtained by the infrared pulse sensor 20 in the reflection principle is an impulse signal S1 that is regarded as weak analog signal. As a result, this signal has to pass through the noise canceling circuit 32 and the filtering shaping amplifying circuit 33 to be converted into a digital signal S2 made available by a microprocessor 51.

The radio frequency module 40 having a wireless signal transmission circuit 41 is provided within the housing 11 for transmitting the heartbeat pulse signal of the filtering shaping amplifying circuit 33 to a display storage unit 50 provided with a wireless signal receiving circuit 42. In this way, the heartbeat pulse signal can be displayed.

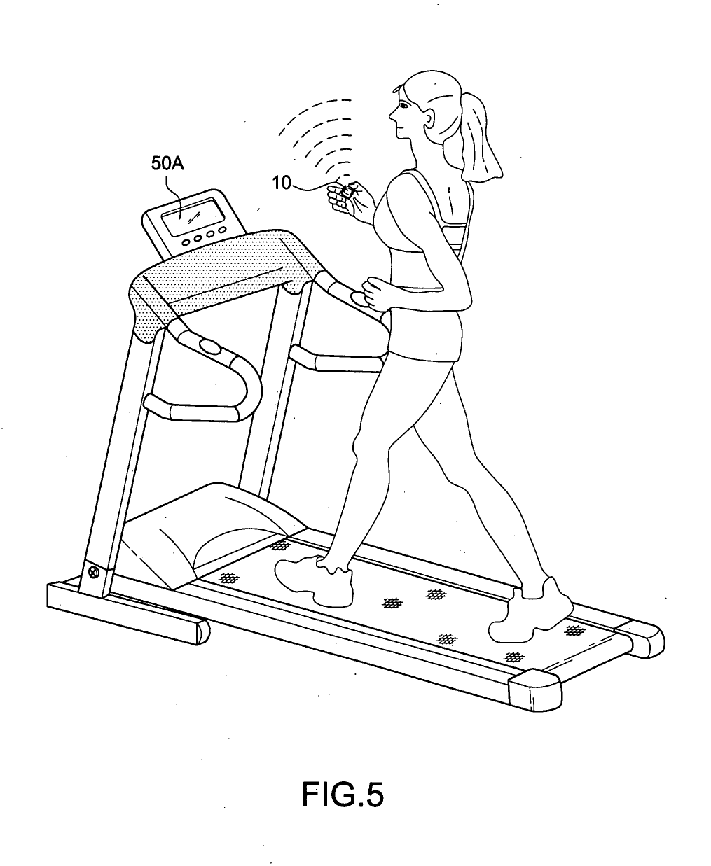

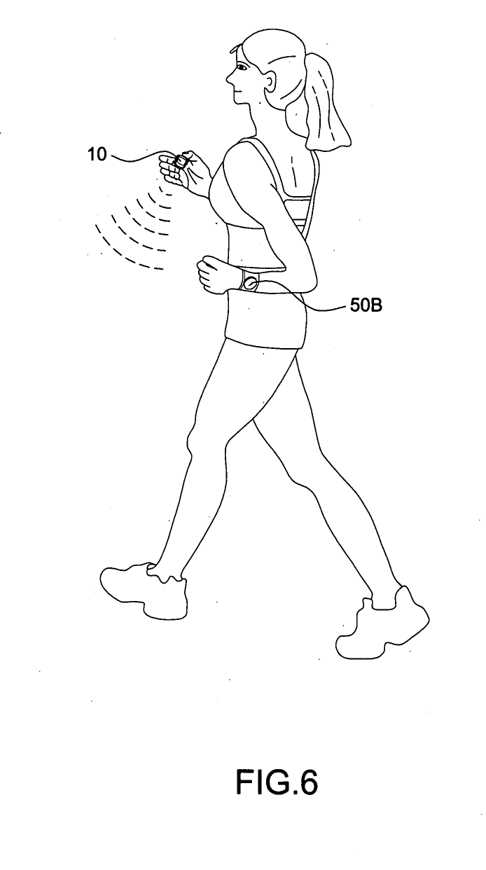

The object of the invention is to reduce the volume and weight of the finger-type pulse detector 10 in such a way that the structure of the invention is minimized in size for easy carrying and use. Meanwhile, a continuous detection of the user's heartbeat takes place during the exercise session. In order to achieve the above-mentioned effect, the housing 11 in accordance with the invention is formed in a finger shape. Moreover, the finger fixing band 14 is employed to bring the infrared pulse sensor 20 at the bottom of the housing 11 in close contact with the finger's skin for detecting the heartbeat value of the user. In accordance with the invention, a periphery electronic product is employed to replace the displaying unit normally occupying more space. In other words, the structure of the invention employs the design of the radio frequency module 40 to transmit the measured heartbeat signal via the wireless signal transmission circuit 41 to the display storage unit 50 with a wireless signal receiving circuit 42. With the rapid development of the technology, most of the current electronic products are provided with display panel such as a console 50A of a sporting equipment (see FIG. 5) and an exercise type wrist watch 50B (see FIG. 6). Besides, the mobile phones, MP3/4 player, etc. also have the function of the display panel. These belong to the prior art so that no further descriptions thereto are given hereinafter. Accordingly, a wireless transmission between the above-mentioned electronic products 50A, 50B and the finger-type pulse detector 10 is achieved only when the wireless signal receiving circuit 42 is installed within the electronic products 50A, 50B.

In addition to the display panel 52, the display storage unit 50 further includes a microprocessor S1, a storage unit 53, and a control switch 54 in which a default program and circuit provided in the microprocessor S1 is used to operate and process the signal inputted by the wireless signal receiving circuit 42 and then to transmit the data to the display panel 52 for a user to read. The storage unit 53 may store the data given by the microprocessor for backup, and the control switch 54 is used to control the display panel 52 to turn ON or OFF for the heart rate data display.

Based on the above-mentioned means, the pulse detector and the heartbeat value displaying unit in accordance with the invention are separated such that the module for measuring the heartbeat is light and in close contact to the blood vessel of the finger, thereby achieving the easy operation and the accurate detection. Meanwhile, the detected signals are wirelessly transmitted to a predetermined display storage unit. Thus, the structure of the invention is flexible and has an expanded range of use.

As shown in FIG. 5, a user takes exercise on a treadmill. The heartbeat signal measured by the finger-type pulse detector 10 on the finger can be transmitted to the console 50A without affecting the exercise of the user.

As shown in FIG. 6, a user takes exercise outdoors. The heartbeat signal measured by the finger-type pulse detector 10 can also be transmitted to the wrist watch 50B. Accordingly, the operation is practical and ergonomic. Meanwhile, the exercise is completely not affected.

While the invention has been described in terms of what is presently considered to be the most practical and preferred embodiment, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

1. A finger-type detection wireless transmission system, including a finger-type pulse detector and a display storage Unit separated therefrom, the finger-type pulse detector comprising:
a) a housing made according to a finger's contour, said housing having a concavely contoured lower surface, a first lug and a second lug being formed at both sides of the housing, an infrared pulse sensor being disposed at the bottom of the housing for detecting the change of a slight amount of blood within the blood vessel of a user's finger that is caused by the pulsation such that the heartbeat pulse signal of the user is available;
b) a button like switch activated by a user disposed at the bottom of the housing for turning power on and off the infrared pulse sensor;
c) a finger fixing band having a first end connected to the first lug and a second end passing through an opening formed through the second lug, the finger fixing band having a pair of displaced fortunes formed on an external surface whereby the finger fixing band is looped trough said second lug opening in a reverse manner for adhering said pair of fasteners each to the other: such tat the housing can be bound to the finger while the infrared pulse sensor and the sensitive switch are in close contact to the finger's skin;
d) a noise-canceling circuit and a filtering shaping amplifying circuit installed within the housing and electrically coupled to the infrared pulse sensor; and
e) a radio frequency module having a wireless signal transmission circuit and provided within the housing for transmitting the heartbeat pulse signal of the filtering shaping amplifying circuit to a display storage unit provided with a wireless signal receiving circuit such that the heartbeat pulse signal can be displayed.

2. The finger-type pulse detection wireless transmission system as recited in claim 1, wherein the infrared pulse sensor includes an infrared transmitting unit disposed on a control circuit board and a receiving unit arranged in parallel thereto.

3. The finger-type pulse detection wireless transmission system recited in claim 1, wherein the display storage unit is a sporting equipment console having the wireless signal receiving circuit and a display panel.

4. The finger-type pulse detection wireless transmission system as recited in claim 1, wherein the display storage unit is a wrist watch having the wireless signal receiving circuit and a display panel.

5. The finger-type pulse detection wireless transmission system as recited in claim 1, wherein the display storage unit further includes a microprocessor, a storage unit, and a control switch, and wherein a default program circuit provided in the microprocessor is used to operate and process the signal and then to transmit the data to the display panel, and wherein the storage unit stores the data given by the microprocessor for backup, and wherein the control switch is used to control the display panel to turn ON or OFF for the heart rate data display.
